(19) United States
(12) Patent Application Publication
(10) Pub. No.: US 2010/0294267 A1
(43) Pub. Date: Nov. 25, 2010

(54) Method and System for Abdominal Weight Loss

(76) Inventor: Stephen M. Terruso, Merrimack, NH (US)

Correspondence Address: HAYES, SOLOWAY P.C., 175 CANAL STREET, MANCHESTER, NH, 03101, US

(21) Appl. No.: 12/784,045

(22) Filed: May 20, 2010

Related U.S. Application Data
(60) Provisional application No. 61/180,202, filed on May 21, 2009.

(51) Int. Cl. A61F 5/24

(52) U.S. Cl. 128/96.1

(57) ABSTRACT

A system and method for abdominal weight loss is provided. An abdominal weight loss apparatus includes a flexible abdominal covering structure sized to cover and fully surround a human abdomen, the abdominal covering structure having a substantially tubular body ending in an upper edge and a lower edge, wherein the abdominal covering structure retains a quantity of body heat proximate to the human abdomen. At least one elastic portion is integral with the abdominal covering structure, wherein the at least one elastic portion retains the abdominal covering structure about the human abdomen.

### CROSS REFERENCE TO RELATED APPLICATION

This application claims benefit of U.S. Provisional Application Ser. No. 61/180,202 filed May 21, 2009, the entire disclosure of which is incorporated herein by reference.

### FIELD OF THE DISCLOSURE

The present disclosure is generally related to weight loss and more particularly is related to a method and system for abdominal weight loss.

### BACKGROUND OF THE DISCLOSURE

Weight loss is a reduction of the total body mass, due to a mean loss of fluid, body fat or adipose tissue and/or lean mass, namely bone mineral deposits, muscle, tendon and other connective tissue. It can occur unintentionally due to medical reasons, but is most frequently seen with a conscious effort to improve an actual or perceived overweight or obese state. Many weight loss methods, systems and tools are available within the art, including various forms of exercising and dieting. However, one's motivation to continue with a weight loss routine may be lessened with time, especially when weight loss results are not visible or do not occur fast enough.

Thus, a heretofore unaddressed need exists in the industry to address the aforementioned deficiencies and inadequacies.

### SUMMARY OF THE DISCLOSURE

Embodiments of the present disclosure provide a method and system for abdominal weight loss. Briefly described, in architecture, one embodiment of the system, among others, can be implemented as follows. The abdominal weight loss apparatus includes a flexible abdominal covering structure sized to cover and fully surround a human abdomen, the abdominal covering structure having a substantially tubular body ending in an upper edge and a lower edge, wherein the abdominal covering structure retains a quantity of body heat proximate to the human abdomen. At least one elastic portion is integral with the abdominal covering structure, wherein the at least one elastic portion retains the abdominal covering structure about the human abdomen.

The present disclosure can also be viewed as providing a method for losing abdominal weight. In this regard, one embodiment of such a method, among others, can be broadly summarized by the following steps: covering and fully surrounding a human abdomen with an abdominal covering structure, the abdominal covering structure having a substantially tubular body ending in an upper edge and a lower edge; retaining a quantity of body heat proximate to the human abdomen with the abdominal covering structure; and retaining the abdominal covering structure about the human abdomen with at least one elastic portion integral with the abdominal covering structure.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a cross-sectional illustration of an abdominal weight loss apparatus, in accordance with a first exemplary embodiment of the present disclosure.

FIG. 2 is a back-view illustration of an abdominal weight loss apparatus, in accordance with the first exemplary embodiment of the present disclosure.

FIG. 3 is a plan view illustration of an abdominal weight loss apparatus, in accordance with the first exemplary embodiment of the present disclosure.

FIG. 4 is a plan view illustration of an abdominal weight loss apparatus, in accordance with the first exemplary embodiment of the present disclosure.

FIG. 5 is a plan view illustration of an abdominal weight loss apparatus, in accordance with a second exemplary embodiment of the present disclosure.

FIG. 6 is a flowchart illustrating a method of losing abdominal weight, in accordance with a third exemplary embodiment of the disclosure.

### DETAILED DESCRIPTION

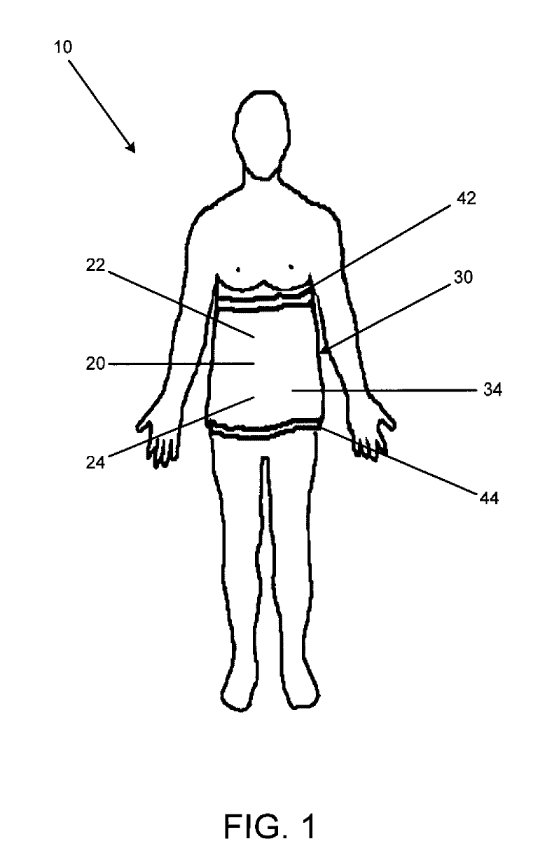

FIG. 1 is an illustration of an abdominal weight loss apparatus 10, in accordance with a first exemplary embodiment of the present disclosure. The abdominal weight loss apparatus 10, hereinafter referred to as apparatus 10, includes a flexible abdominal covering structure 20 sized to cover and fully surround a human abdomen 30. The abdominal covering structure 20 has a substantially tubular body ending in an upper edge 22 and a lower edge 24. The abdominal covering structure 20 retains a quantity of body heat proximate to the human abdomen 30. At least two elastic portions are integral with the abdominal covering structure 20, including a first elastic portion 42 located proximate to the upper edge 22, and a second elastic portion located proximate to the lower edge 24. The first and second elastic portions 42, 44 retain the abdominal covering structure 20 about the human abdomen 30.

As is shown in FIG. 1, the abdominal covering structure 20 may to cover and fully surround a human abdomen 30, including the human belly 34. The human abdomen 30 may be any size, including a small size, a very large size, or any size in between. To fit accordingly, the abdominal covering structure 20 may also be any overall size, any length, width or thickness. The abdominal covering structure 20 may be constructed from a heat retention material such as a polyvinyl chloride (PVC) material. For example, the abdominal covering structure 20 may be constructed from a 4 mm PVC material, although any thickness of material may be used.

The heat retention material may retain the body heat from the human abdomen 30 proximate to the human abdomen 30. This may result in a high temperature at the human abdomen 30 with the abdominal covering structure 20, than without the abdominal covering structure 20 during any physical activity. The physical activity may include any type of activity, such as running, jogging, exercising, walking, or otherwise moving. In use during a physical activity, the abdominal covering structure 20 will retain the body heat of the human abdomen 30, thereby raising the temperature of the human abdomen 30. This will result in an increase of perspiration, which subsequently results in weight loss within the human abdomen 30.

The abdominal covering structure 20 also includes at least two elastic portions integral with the abdominal covering structure 20. These elastic portions may be sewn into or otherwise affixed to the abdominal covering structure 20. The first elastic portion 42 located proximate to the upper edge 22. As shown, the upper edge 22 may be proximate to a human chest, generally situated proximate to the lower portion of a human rib cage. The second elastic portion is located proximate to the lower edge 24 of the abdominal covering structure 20, which is proximate to a waist line of the human. The first and second elastic portions 42, 44 retain the abdominal covering structure 20 about the human abdomen 30 by elastically securing the abdominal covering structure 20 over the human abdomen 30. The first and second elastic portions 42, 44 may be any size to fit with any size human abdomen 30. Additional elastic portions may also be included at any other location in the abdominal covering structure 20.

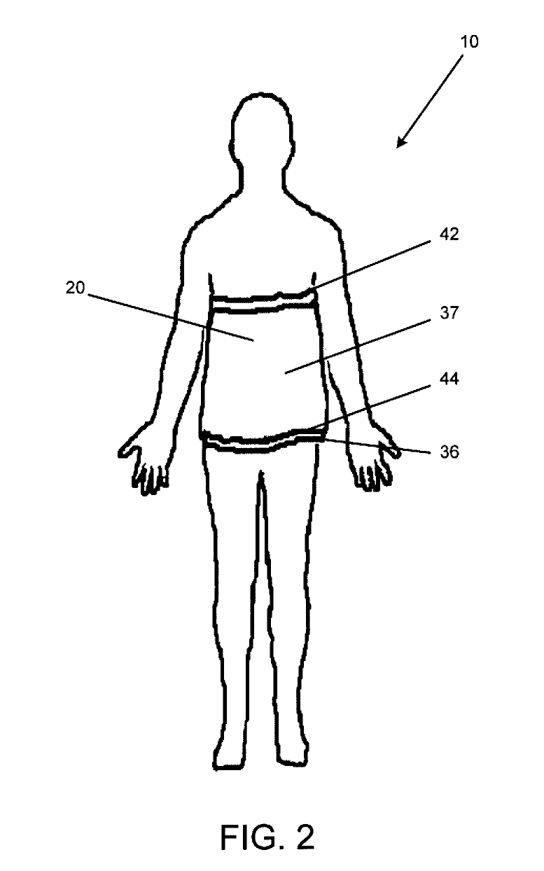

FIG. 2 is a back-view illustration of an abdominal weight loss apparatus 10, in accordance with the first exemplary embodiment of the present disclosure. The abdominal covering structure 20 may fully surround the human abdomen 30 which includes a human lower back 32, a human belly 34 (FIG. 1) and a human abdominal external oblique muscle 36, commonly referred to as the ‘love handles’. The abdominal covering structure 20 may be placed on the human abdomen 30 by slipping the abdominal covering structure 20 over the human's head, or by stepping into the abdominal covering structure 20 and pulling the abdominal covering structure 20 up to the human abdomen 30. In addition, the abdominal covering structure 20 may also be placed on the human abdomen 30 with a connecting element, such as a zipper, a hook and loop fastener system or one or more buttons, located within the abdominal covering structure 20.

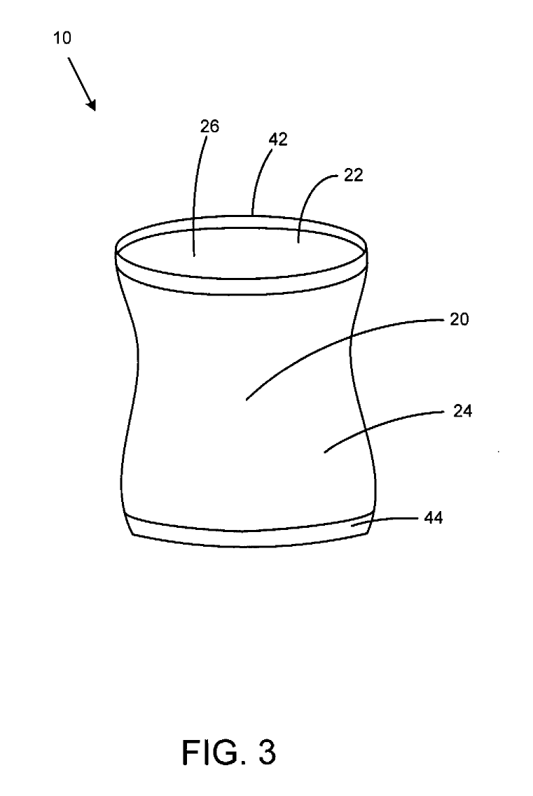

FIG. 3 is a plan view illustration of an abdominal weight loss apparatus 10, in accordance with the first exemplary embodiment of the present disclosure. As is shown, the abdominal covering structure 20 may be a tubular structure, having an interior portion 26. An external or internal surface of the abdominal covering structure 20 may be any color, preferably a color that substantially matches at least one article of clothing on the human abdomen 30 (FIGS. 1 & 2). This may decrease the visibility of the abdominal covering structure 20 when it is worn under an article of clothing or help hide the appearance of the abdominal covering structure 20 when it is worn under clothing. During a physical activity, the user of the abdominal covering structure 20 may tuck the lower edge 24 of the abdominal covering structure 20 into their pants or gym shorts.

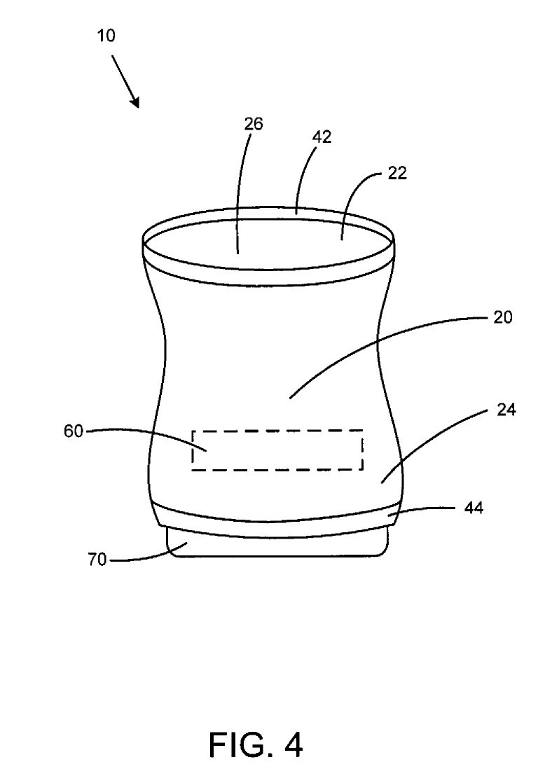

FIG. 4 is a plan view illustration of an abdominal weight loss apparatus 10, in accordance with the first exemplary embodiment of the present disclosure. The abdominal covering structure 20 may include at least one electric heating element 60 integral with the abdominal covering apparatus 20. The electric heating element 60 may provide additional heat to the human abdomen 30, thereby further increasing the temperature of the human abdomen 30. Any type of electric heating element 60 may be used. Likewise, a non-electric heating source may be used. The abdominal covering structure 20 may also include at least one absorption pad 70, constructed from any absorption material, and located proximate to the abdominal covering structure 20 for soaking up a quantity of human perspiration. The absorption pad 70 may be located at any position within the abdominal covering structure 20 and may be removable, disposable and/or washable. The abdominal covering structure 20 may also be sized to cover and fully surround another body part beyond the human abdomen 30. This may include covering and fully surrounding a human arm, a human leg, a human thigh and/or a human buttock. As one having skill in the art will recognize, the abdominal covering apparatus 10 may be used in a variety of different locations on the human body.

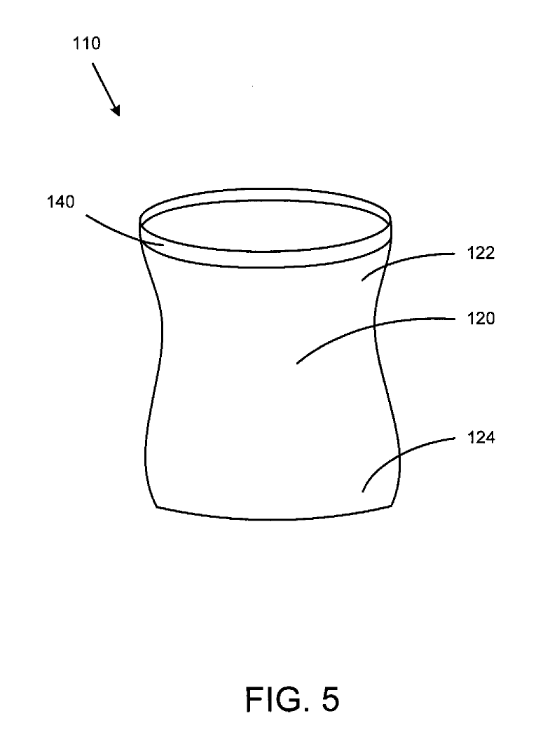

FIG. 5 is a plan view illustration of an abdominal weight loss apparatus 110, in accordance with a second exemplary embodiment of the present disclosure. The abdominal weight loss apparatus 110 of the second exemplary embodiment is substantially similar to the abdominal weight loss apparatus 10 of the first exemplary embodiment, and may include any of the features and components disclosed within the first exemplary embodiment. The abdominal weight loss apparatus 110 includes a flexible abdominal covering structure 120 sized to cover and fully surround a human abdomen (not shown). The abdominal covering structure 120 has a substantially tubular body ending in an upper edge 122 and a lower edge 124. The abdominal covering structure 120 retains a quantity of body heat proximate to the human abdomen. At least one elastic portion 140 is integral with the abdominal covering structure 120 and retains the abdominal covering structure 120 about the human abdomen.

The at least one elastic portion 140 may be located at any position within the abdominal covering structure 120, such as proximate to one of the upper edge 122 and the lower edge 124. The at least one elastic portion 140 may also be integral within the entire abdominal covering structure 120, such as when the abdominal covering structure 120 is constructed from an elastic material. In FIG. 5, the at least one elastic portion 140 is shown as being proximate to the upper edge 122. The abdominal weight loss apparatus 110 may include any number of additional features, including those discussed with reference to the first exemplary embodiment, all of which are considered within the scope of the present disclosure.

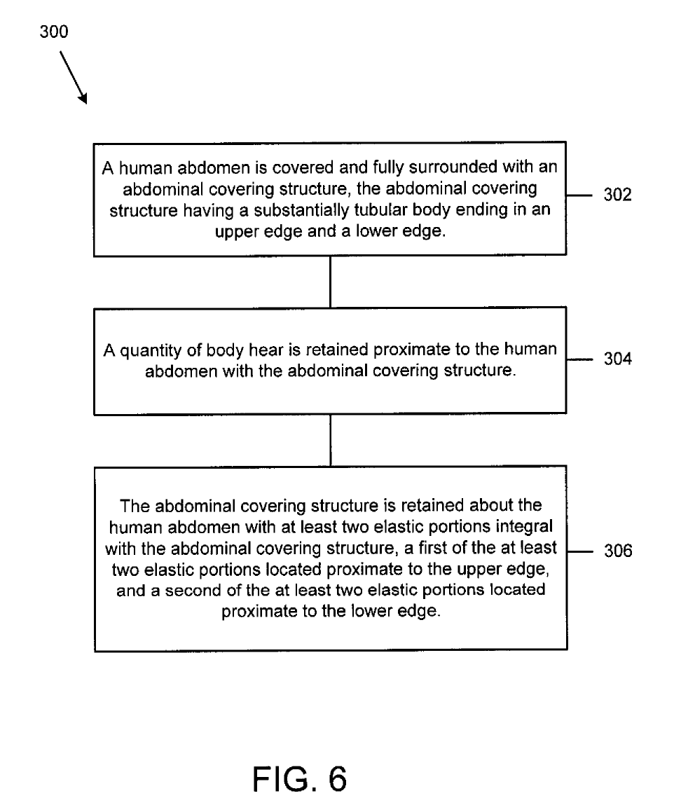

FIG. 6 is a flowchart 300 illustrating a method of losing abdominal weight, in accordance with a third exemplary embodiment of the disclosure. It should be noted that any process descriptions or blocks in flow charts should be understood as representing modules, segments, portions of code, or steps that include one or more instructions for implementing specific logical functions in the process, and alternate implementations are included within the scope of the present disclosure in which functions may be executed out of order from that shown or discussed, including substantially concurrently or in reverse order, depending on the functionality involved, as would be understood by those reasonably skilled in the art of the present disclosure.

As is shown by block 302 a human abdomen is covered and fully surrounded with an abdominal covering structure, the abdominal covering structure having a substantially tubular body ending in an upper edge and a lower edge. A quantity of body heat is retained proximate to the human abdomen with the abdominal covering structure (block 304). The abdominal covering structure is retained about the human abdomen with at least two elastic portions integral with the abdominal covering structure, a first of the at least two elastic portions located proximate to the upper edge, and a second of the at least two elastic portions located proximate to the lower edge (block 306). Additional steps may also be included, such as the step of engaging the human abdomen in a physical activity, which may include jogging, running, walking, or any other physical activity. Furthermore, a quantity of human perspiration may be collected with at least one absorption pad proximate to the abdominal covering structure.

It should be emphasized that the above-described embodiments of the present disclosure, particularly, any “preferred” embodiments, are merely possible examples of implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and the present disclosure and protected by the following claims.

What is claimed is:

1. An abdominal weight loss apparatus comprising:
a flexible abdominal covering structure sized to cover and fully surround a human abdomen, the abdominal covering structure having a substantially tubular body ending in an upper edge and a lower edge, wherein the abdominal covering structure retains a quantity of body heat proximate to the human abdomen; and
at least one elastic portion integral with the abdominal covering structure, wherein the at least one elastic portion retains the abdominal covering structure about the human abdomen

2. The abdominal weight loss apparatus of claim 1, wherein the at least one elastic portion is located proximate to one of the upper edge and the lower edge.

3. The abdominal weight loss apparatus of claim 1, wherein the at least one elastic portion further comprises a first elastic portion and a second elastic portion, wherein the first elastic portion is located proximate to the upper edge and the second elastic portion is located proximate to the lower edge, wherein the first and second elastic portions retain the abdominal covering structure about the human abdomen.

4. The abdominal weight loss apparatus of claim 1, wherein the abdominal covering structure is constructed from a polyvinyl chloride material.

5. The abdominal weight loss apparatus of claim 1, wherein the abdominal covering structure fully surrounds at least one of a human lower back, a human belly and a human abdominal external oblique muscle.

6. The abdominal weight loss apparatus of claim 1, wherein the abdominal weight loss apparatus has an external color that substantially matches at least one article of clothing on the human abdomen.

7. The abdominal weight loss apparatus of claim 1, wherein the lower edge of the abdominal covering structure is tucked within a pair of pants.

8. The abdominal weight loss apparatus of claim 1, further comprising at least one absorption pad proximate to the abdominal covering structure, wherein the at least one absorption pad soaks up a quantity of human perspiration.

9. The abdominal weight loss apparatus of claim 1, wherein the abdominal covering structure is sized to cover and fully surround at least one of a human arm, a human leg, a human thigh and a human buttock.

10. A method for losing abdominal weight comprising the steps of:
covering and fully surrounding a human abdomen with an abdominal covering structure, the abdominal covering structure having a substantially tubular body ending in an upper edge and a lower edge;
retaining a quantity of body heat proximate to the human abdomen with the abdominal covering structure; and
retaining the abdominal covering structure about the human abdomen with at least one elastic portion integral with the abdominal covering structure.

11. The method for losing abdominal weight of claim 10, wherein the at least one elastic portion further comprises a first elastic portion located proximate to the upper edge and a second elastic portion located proximate to the lower edge.

12. The method for losing abdominal weight of claim 10, further comprising the step of engaging the human abdomen in a physical activity.

13. The method for losing abdominal weight of claim 10, further comprising the step of collecting a quantity of human perspiration with at least one absorption pad proximate to the abdominal covering structure.
